(19) United States
(12) Patent Application Publication
(10) Pub. No.: US 2020/0155766 A1
(43) Pub. Date: May 21, 2020

(54) PRE-FILLED SYRINGE CONTAINING MOXIFLOXACIN

(71) Applicant: NAL PHARMACEUTICAL GROUP LIMITED, Causeway Bay (HK)

(72) Inventors: Y. Joseph MO, Princeton, MO (US); Xudong YUAN, Morganville, NJ (US)

(73) Assignee: NAL PHARMACEUTICAL GROUP LIMITED, Causeway Bay (HK)

(21) Appl. No.: 16/604,486

(22) PCT Filed: Apr. 11, 2018
(86) PCT No.: PCT/US2018/027072

§ 371 Date: Oct. 10, 2019

Related U.S. Application Data
(60) Provisional application No. 62/485,519, filed on Apr. 14, 2017.

(51) Int. Cl. A61M 5/315; A61K 9/00; A61K 9/08; A61K 31/4709; A61M 5/31

(52) U.S. Cl. CPC A61M 5/31533; A61K 9/0048; A61K 9/08; A61M 2210/0612; A61M 5/3129; A61M 2005/3125; A61K 31/4709

(57) ABSTRACT

The present invention relates to a pre-filled syringe containing Moxifloxacin has a plunger, a barrel, a needle with gauge, kits having this syringe and the use of the syringe for the administration of Moxifloxacin for postsurgical bacterial endophthalmitis after cataract surgery.

### CROSS REFERENCE TO RELATED APPLICATION

This application claims the benefit of U.S. provisional patent application Ser. No. 62/485,519, filed Apr. 14, 2017, the contents of which are incorporated herein by reference.

### FIELD OF THE INVENTION

The present invention relates to a pre-filled syringe containing Moxifloxacin and comprising a plunger, a barrel, a needle with gauge, kits comprising this syringe and the use of the syringe for the administration of Moxifloxacin intracamerally to prevent postsurgical bacterial endophthalmitis post cataract surgery. [symbol] p.

### BACKGROUND OF THE INVENTION

Endophthalmitis is an inflammation of the internal coats of the eye, potentially a devastating complication of cataract surgery that occurs in 0.04% to 0.2% of the cases.

Taking steps to minimize the occurrence of endophthalmitis has always been a part of cataract surgery. Historically, most options for chemoprophylaxis against bacterial endophthalmitis have been topical and subconjunctival antibiotics and povidone-iodine preparation and instillation on the ocular surface before surgery. In the United States, the most common form of chemoprophylaxis has been topical fluoroquinolones prescribed 1 to 3 days postoperatively and resumed immediately postoperatively for one week. Most European surgeons now favor intracameral antibiotic injection over topical antibiotics or subconjunctival antibiotics alone. Increasing evidence supports the use of intracameral injection to prevent bacterial endophthalmitis has higher efficacy than topical administration. However, it is considered off-label to inject antibiotics intracamerally in the US.

Moxifloxacin, marketed under the name Vigamox®, is a fluoroquinolone antibiotic agent eye drop used to treat bacterial infection of the eyes. Vigamox® is presented in solution form in a bottle with 5 mg/ml Moxifloxacin concentration. In the US, most surgeons had their patients apply Vigamox® topically pre and post cataract operation for postsurgical endophthalmitis prophylaxis. Off-label use of Vigamox® directed for endophthalmitis prophylaxis is also performed in the US. Vigamox® is extracted with syringe from bottle for intracameral injection after cataract surgery.

The present invention of pre-filled syringe has many benefits compared to a bottle and a separately prepared syringe, such as improved safety, affordability, convenience, accuracy and sterility. The use of pre-filled syringes results in greater dose precision, in a reduction of the potential for needle sticks injuries that can occur while drawing medication from bottles, in pre-measured dosage reducing dosing errors and the risk of contamination due to the need to reconstitute and/or draw medication into a syringe, and in less overfilling of the syringe helping to reduce costs by minimizing drug waste. Moxifloxacin may have advantages as a first-line drug than other fluoroquinolone antibiotic agent, because Moxifloxacin offers boarder spectrum coverage and is available for injection without dilution.

Hence, there is an unmet medical need for Moxifloxacin pre-filled syringes which can safely deliver the drug to the eye and which have the above benefits, but in which the drug is stable for the storage period.

### SUMMARY OF THE INVENTION

It has been demonstrated that Moxifloxacin solution retains potency, sterility and stability during storage when filled into a pre-filled polypropylene syringe (Armstrong et al., 2010). The pre-filled syringe of the present invention does not contain a significant amount of particles. The present invention provides a pre-filled syringe containing a liquid formulation of Moxifloxacin and comprising a syringe barrel, wherein the syringe barrel is preferably made of plastic/glass and is silicone-free.

Moxifloxacin is a fluoroquinolone antibiotic agent, the chemical classification of Moxifloxacin is Quinolones. Moxifloxacin binds to and inhibits the bacterial enzymes DNA gyrase and topoisomerase IV, resulting in inhibition of DNA replication and repair, and cell death in sensitive bacterial species.

In a certain embodiment of the present invention, the Moxifloxacin concentration is 0.15 to 500 mg/ml. In one aspect of the invention the pre-filled syringe contains less than 50 particles per ml of the liquid formulation having a diameter of 10 μm or greater.

In another aspect of the invention, the pre-filled syringe contains less than 5 particles per ml of the liquid formulation having a diameter of 25 μm or greater. In still another aspect of the invention, the pre-filled syringe has a gliding force of less than or equal to about 10N.

In a preferred embodiment, the pre-filled syringe further comprises a silicone-free stopper.

Preferably, the syringe barrel is made of cycloolefin polymer or cycloolefin copolymer. In a preferred embodiment the syringe barrel comprises an internal coating other than a silicone coating. Also preferably, the pre-filled syringe comprises a staked needle.

The present invention also provides a kit comprising one or more pre-filled syringes according to the present invention. Preferably, the kit is a blister pack.

The pre-filled syringe may be used in administering Moxifloxacin intracamerally to a patient post cataract surgery for postsurgical endophthalmitis prophylaxis.

Preferably, a volume of 1 to 500 μL of the liquid formulation is administered to the patient.

### BRIEF DESCRIPTION OF THE DRAWINGS

FIG. 1 shows an example of a pre-filled syringe of the present invention for intracameral injection of Moxifloxacin. The pre-filled syringe composes of plunger head, plunger, needle, needle cap, needle guard activation clip, needle guard wings, label, syringe barrel, viewing window, dose marking.

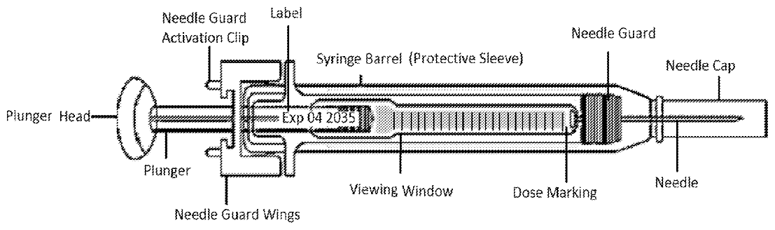

### DETAILED DESCRIPTION OF THE INVENTION

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention. Unless indicated otherwise, all ingredient concentrations are presented in units of % weight/volume % w/v).

Moxifloxacin is preferably present in the compositions of the present invention in the form of a pharmaceutically acceptable salt. Most preferably, moxifloxacin is present in the form of moxifloxacin hydrochloride. The compositions contain moxifloxacin in an amount equivalent to about 0.5% as the free base. The amount of moxifloxacin hydrochloride in the compositions of the present invention is 0.5-0.6%, and is most preferably 0.545%, which is equivalent to 0.5% moxifloxacin as base.

The compositions of the present invention may contain boric acid in an amount from 0.2-0.4%, preferably 0.3%.

In a certain embodiment of the present invention, Edetate &sodium is present in the compositions of the present invention in an amount of 0.005-0.02%. Most preferably, the edetate disodium is present in an amount of 0.01%.

An ionic tonicity adjusting agent is added to the compositions of the present invention in an amount sufficient to cause the final composition to have an osmolality of 270-330 mOsm/Kg. Preferably, the ionic tonicity adjusting agent is sodium chloride and is present in an amount of 0.5-0.8%. Most preferably, the compositions of the present invention contain 0.65% NaCl.

The compositions of the present invention further contain an otically and ophthalmically acceptable non-ionic surfactant, such as a polysorbate surfactant, a block copolymer of ethylene oxide and propylene oxide surfactant (e.g., a pluronic or tetronic surfactant), or tyloxapol. Preferably, the compositions contain the non-ionic surfactant in an amount of 0.04-0.06%. Most preferably, the non-ionic surfactant is tyloxapol and the amount of tyloxapol in the compositions of the present invention is 0.05%.

The compositions can contain a preservative ingredient or a preservation-enhancing ingredient selected from the group consisting of benzalkonium chloride and sorbitol. Preferably, the compositions of the present invention contain benzalkonium chloride if they are intended for topical otic administration and sorbitol if they are intended for topical ophthalmic administration. If present, the amount of benzalkonium chloride in the compositions is 0.005-0.015%, preferably 0.01%. If present, the amount of sorbitol in the compositions of the present invention is 0.1-0.3%, preferably 0.2%. In addition, the compositions can be preservative free and sterile formulation.

The pH of the aqueous solutions of the present invention is adjusted with an ophthalmically acceptable pH-adjusting agent. Ophthalmically acceptable pH adjusting agents are known and include, but are not limited to, hydrochloric acid (HCl) and sodium hydroxide (NaOH). The compositions of the present invention preferably contain NaOH or HCl to obtain the desired pH. The compositions of the present invention are formulated and maintained within a narrow pH range in order to keep the compositions stable over a commercially acceptable shelf-life period. The compositions of the present invention have a pH of 5.0-9.0, and most preferably 7.8-8.0.

The compositions of the present invention are preferably packaged in prefilled syringes or multi-dose plastic containers designed to deliver drops to the eye. Preferably pre-filled syringe containing a liquid formulation of Moxifloxacin and comprising a syringe barrel, wherein the syringe barrel is made of plastic/glass and is silicone-free.

Preferably, the pre-filled syringe is a disposable and for one dose to avoid contamination.

### EXAMPLES

The following examples are intended to illustrate, but not limit, the present invention. While the invention has been described with respect to the specific embodiments, it will be apparent to those skilled in the art that various changes and modifications may be made without departing from the spirit and scope of the invention as defined in the listed claims.

### Example 1. Ophthalmic Solution Containing Moxifloxacin Hydrochloride Eq. to Moxifloxacin (0.3% w/v Dissolved) in Prefilled Syringe

TABLE 1

| No. | Pharmaceutical Ingredients | Quantities (%) |
|---|---|---|
| 1 | Moxifloxacin Hydrochloride | 0.3 |
| 2 | Benzalkonium Chloride | 0.01 |
| 3 | Boric acid | 0.2 |
| 4 | Mannitol | 4.0 |
| 5 | EDTA | 0.01 |
| 6 | Hydrochloric Acid and/or | pH adjustment to |
| | Sodium Hydroxide, NF | 5.5 |
| 7 | Water for injection | q.s. to 100 |

A formulation as shown in table 1 is prepared as follows:

(a) Accurately weigh quantity of Moxifloxacin Hydrochloride and required quantity of Disodium edetate (EDTA) are introduced into suitable container and dissolved it in sufficient water for injection and stirred until clear colorless solution is obtained.

(b) Required quantity of Boric acid and Mannitol are dissolved in sufficient quantity of water for injection in separate container and stirred until clear solution is obtained; this solution is added to solution of step (a) with stirring.

(c) Required quantity of Benzalkonium Chloride is dissolved in sufficient quantity of water for injection in separate container and stirred until clear solution is obtained; this solution is added to solution of step (a) with stirring to obtain a final solution.

(d) The pH of final solution obtained as per step (c) is adjusted to 5.5 with required quantity of 1 N NaOH or 0.1 N HCL stock solution and final volume of desired batch size is made up with sufficient quantity of water for injection.

(e) The solution of step (d) is then filtered through 0.22 μm sterile filter.

(f) The filtered sterilized Moxifloxacin solution of step (e) is used to fill the prefilled syringes.

Example 2. Ophthalmic Solution Containing Moxifloxacin Hydrochloride Eq. to Moxifloxacin (0.5% w/v Dissolved) in Prefilled Syringe

TABLE 2

| No. | Pharmaceutical Ingredients | Quantities (%) |
|---|---|---|
| 1 | Moxifloxacin Hydrochloride | 0.5 |
| 2 | Benzalkonium Chloride | 0.01 |
| 3 | Boric acid | 0.1 |
| 4 | Sodium Chloride | 0.8 |
| 5 | EDTA | 0.01 |
| 6 | Hydrochloric Acid and/or | pH adjustment to |
| | Sodium Hydroxide, NF | 6.5 |
| 7 | Water for injection | q.s. to 100 |

A formulation as shown in table 2 is prepared as follows:

(a) Accurately weigh quantity of Moxifloxacin Hydrochloride and required quantity of Disodium edetate (EDTA) are introduced into suitable container and dissolved it in sufficient water for injection and stirred until clear colorless solution is obtained.

(b) Required quantity of Boric acid and Sodium Chloride are dissolved in sufficient quantity of water for injection in separate container and stirred until clear solution is obtained; this solution is added to solution of step (a) with stirring.

(c) Required quantity of Benzalkonium Chloride is dissolved in sufficient quantity of water for injection in separate container and stirred until clear solution is obtained; this solution is added to solution of step (a) with stirring to obtain a final solution.

(d) The pH of final solution obtained as per step (c) is adjusted to 6.5 with required quantity of 1 N NaOH or 0.1 N HCL stock solution and final volume of desired batch size is made up with sufficient quantity of water for injection.

(e) The solution of step (d) is then filtered through 0.2.2 μm sterile filter.

(f) The filtered sterilized Moxifloxacin solution of step (e) is used to fill the prefilled syringes.

### Example 3. Ophthalmic Solution Containing Moxifloxacin Hydrochloride Eq. to Moxifloxacin (0.6% w/v Dissolved) in Prefilled Syringe

TABLE 3

| No. | Pharmaceutical Ingredients | Quantities (%) |
|---|---|---|
| 1 | Moxifloxacin Hydrochloride | 0.6 |
| 2 | Benzalkonium Chloride | 0.01 |
| 3 | Boric acid | 0.1 |
| 4 | Sodium Chloride | 0.7 |
| 5 | Sorbitol | 0.1 |
| 5 | EDTA | 0.01 |
| 6 | Hydrochloric Acid and/or | pH adjustment to |
| | Sodium Hydroxide, NF | 7.5 |
| 7 | Water for injection | q.s. to 100 |

A formulation as shown in table 3 is prepared as follows:

(a) Accurately weigh quantity of Moxifloxacin Hydrochloride and required quantity of Disodium edetate (EDTA) are introduced into suitable container and dissolved it in sufficient water for injection and stirred until clear colorless solution is obtained.

(b) Required quantity of Boric acid and Sodium Chloride and Sorbitol are dissolved in sufficient quantity of water for injection in separate container and stirred until clear solution is obtained; this solution is added to solution of step (a) with stirring.

(c) Required quantity of Benzalkonium Chloride is dissolved in sufficient quantity of water for injection in separate container and stirred until clear solution is obtained; this solution is added to solution of step (a) with stirring to obtain a final solution.

(d) The pH of final solution obtained as per step (c) is adjusted to 7.5 with required quantity of 1 N NaOH or 0.1 N HCL stock solution and final volume of desired batch size is made up with sufficient quantity of water for injection.

(e) The solution of step (d) is then filtered through 0.22 μm sterile filter.

(f) The filtered sterilized Moxifloxacin solution of step (e) is used to fill the prefilled syringes.

### Example 4. Preservative Free Ophthalmic Solution Containing Moxifloxacin Hydrochloride Eq. to Moxifloxacin (0.5% w/v Dissolved) in Prefilled Syringe

TABLE 4

| No. | Pharmaceutical Ingredients | Quantities (%) |
|---|---|---|
| 1 | Moxifloxacin Hydrochloride | 0.5 |
| 2 | Boric acid | 0.1 |
| 3 | Sodium Chloride | 0.8 |
| 4 | Hydrochloric Acid and/or | pH adjustment to |
| | Sodium Hydroxide, NF | 7.5 |
| 5 | Water far injection | q.s. to 100 |

A formulation as shown in table 4 is prepared as follows:

(a) Accurately weigh quantity of Moxifloxacin Hydrochloride is introduced into suitable container and dissolved it in sufficient water for injection and stirred until clear colorless solution is obtained.

(b) Required quantity of Boric acid and Sodium Chloride are dissolved in sufficient quantity of water for injection in separate container and stirred until clear solution is obtained; this solution is added to solution of step (a) with stirring.

(c) The pH of final solution obtained as per step (b) is adjusted to 7.5 with required quantity of 1 N NaOH or 0.1 N HCL stock solution and the final volume of desired hatch size is made up with sufficient quantity of water for injection.

(d) The solution of step (c) is then filtered through 0.22 μm sterile filter.

(e) The filtered sterilized Moxifloxacin solution of step (d) is used to fill the prefilled syringes.

### Example 5. Preservative Free Ophthalmic Solution Containing Moxifloxacin Hydrochloride Eq. to Moxifloxacin (0.6% w/v Dissolved) in Prefilled Syringe

TABLE 5

| No. | Pharmaceutical Ingredients | Quantities (%) |
|---|---|---|
| 1 | Moxifloxacin Hydrochloride | 0.6 |
| 2 | Sodium Chloride | 0.8 |
| 3 | Hydrochloric Acid and/or | pH adjustment to |
| | Sodium Hydroxide, NF | 7.5 |
| 4 | Water for injection | q.s. to 100 |

A formulation as shown in table 5 is prepared as follows:

(a) Accurately weigh quantity of Moxifloxacin Hydrochloride is introduced into suitable container and dissolved it in sufficient water for injection and stirred until clear colorless solution is obtained.

(b) Required quantity of Boric acid and Sodium Chloride are dissolved in sufficient quantity of water for injection in separate container and stirred until clear solution is obtained; this solution is added to solution of step (a) with stirring.

(c) The pH of final solution obtained as per step (b) is adjusted to 7.5 with required quantity of 1 N NaOH or 0.1 N HCL stock solution and final volume of desired batch size is made up with sufficient quantity of water for injection.

(d) The solution of step (c) is then filtered through 0.22 μm sterile filter.

(e) The filtered sterilized Moxifloxacin solution of step (d) is used to fill the prefilled syringes. Example 6. Determination of Particles of Different Sizes

The particle size of particles in different plastic and glass syringes containing Moxifloxacin solution and subjected to different conditions is determined by a Horiba laser light scattering particle sizer. The appropriate amount of Moxifloxacin solution is transferred from the prefilled syringes to liquid sampling cell and the particle sizes and distribution are determined by the particle sizer.

### Example 7. Determination of Moxifloxacin Stability

Samples in different plastic and glass prefilled syringes are subjected to stress conditions to study the stability of Moxifloxacin formulation. The following storage condition are used: 4° C./35% RH, 25° C./40% RH, 25° C./40% RH Horizontal, 30° C/65% RH, 40° C./<25% RH, freeze-thaw cycle. Each cycle consisted of 24 hours at −20° C. followed by 24 hours at room temperature.

### Example 8. Determination of Gliding Forces in Different Plastic and Glass Syringes Containing Moxifloxacin Solution

The glide force (F) can be determined by the following equation:

F=[Øηl/πr⁴]* A

where A is the area of barrel lumen; r is radius of tube; η is viscosity; Ø is the laminar flow through a tube (Poiseuille's Law):

Ø=[πr⁴/8η]*[(ΔP)/l]

where ΔP is the pressure differential; and l is the length of the tube.

The gliding force and break loose force can be determined by a Tensile Compression Testing Machine. The relationship between compression load (N) and compression extension (mm) can be plotted after measurement. The break loose force and gliding force can be therefore determined from the plot. The syringeability can be optimized based on the break loose force and gliding force.

1-15. (canceled)

16. A pre-filled syringe containing a liquid formulation of Moxifloxacin and comprising a syringe barrel, wherein the syringe barrel is made of plastic or glass and is silicone-free.

17. The pre-filled syringe of claim 16, wherein Moxifloxacin concentration is 0.1 to 500 mg/ml.

18. The pre-filled syringe of claim 16, containing less than 50 particles per ml of the liquid formulation having a diameter of 10 μm or greater.

19. The pre-filled syringe of claim 16, containing less than 5 particles per ml of the liquid formulation having a diameter of 25 μm or greater.

20. The pre-filled syringe of claim 16, having a gliding force of less than or equal to about 10N.

21. The pre-filled syringe of claim 16, further comprising a a silicone-free stopper.

22. The pre-filled syringe of claim 16, wherein the syringe barrel is made of polymer, cycloolefin, or glass.

23. The pre-filled syringe of claim 16, wherein the syringe barrel comprises an internal coating other than a silicone coating.

24. The pre-filled syringe according to claim 16, further comprising a staked needle.

25. A kit comprising the pre-filled syringe of claim 16.

26. A method for treating postsurgical endophthalmitis prophylaxis or other related eye diseases and conditions comprising:
administering a liquid formulation of Moxifloxacin in the pre-filled syringe of claim 16 to a patient with postsurgical endophthalmitis prophylaxis or other related eye diseases and conditions.

27. The method of claim 26, wherein a volume of 1 to 500 μm of the liquid formulation is administered to the patient by intracameral injection into the anterior chamber.
